(19) United States
(12) Patent Application Publication
(10) Pub. No.: US 2010/0240594 A1
(43) Pub. Date: Sep. 23, 2010

(54) TARGETED DELIVERY OF CHEMOTHERAPEUTIC AGENTS

(75) Inventor: Maurizio Pellecchia, San Diego, CA (US)

Correspondence Address: DLA PIPER LLP (US), 4365 EXECUTIVE DRIVE, SUITE 1100, SAN DIEGO, CA, 92121-2133, US

(73) Assignee: Burnham Institute for Medical Research, La Jolla, CA (US)

(21) Appl. No.: 12/728,077

(22) Filed: Mar. 19, 2010

Related U.S. Application Data
(60) Provisional application No. 61/162,176, filed on Mar. 20, 2009.

(51) Int. Cl. A61K 38/10; C07K 7/08; C07K 1/113; A61P 35/00

(52) U.S. Cl. 514/19.3; 530/327; 514/21.5; 530/345

(57) ABSTRACT

The disclosure provides compounds and compositions, and methods of using these compounds and compositions, for the targeted delivery of chemotherapeutic agents.

### CROSS REFERENCE TO RELATED APPLICATIONS

This application claims the benefit of priority under 35 U.S.C. §119(e) to U.S. Provisional Application No. 61/162,176, filed Mar. 20, 2009, the disclosure of which is hereby incorporated by reference in its entirety for all purposes.

### BACKGROUND OF THE DISCLOSURE

1. Field of the Disclosure

The disclosure generally relates to compounds and compositions, and methods of using these compounds and compositions, for the targeted delivery of chemotherapeutic agents useful for treating cancer.

2. Background Information

Eph receptors and ephrins, their corresponding ligands, are components of cell signaling pathways involved in animal development and are implicated in some cancers. Eph receptors are classified as receptor tyrosine kinases (RTKs), which form the largest sub-family of RTKs. There are 16 known Eph receptors (14 found in mammals): EPHA1, EPHA2, EPHA3, EPHA4, EPHA5, EPHA6, EPHA7, EPHA8, EPHA9, EPHA10, EPHB1, EPHB2, EPHB3, EPHB4, EPHB5, and EPHB6; and 9 known ephrin ligands (8 found in mammals): EFNA1, EFNA2, EFNA3, EFNA4, EFNA5, EFNB1, EFNB2, and EFNB3. The Eph receptors influence cell shape and migration by acting directly on the actin cytoskeleton. In addition, they can affect cell proliferation and survival. Post-translational phosphorylation of proteins is an important regulatory mechanism in cell function and interaction. Its modification and consequent receptor/ligand alteration is thus one of the many factors that might result in the transformation of normal cells into an invasive or metastatic tumor. In view of their pivotal role in cell-migration and cell-cell interaction, it is not surprising that Eph receptors and ephrins affect tumor growth and cancer cell metastasis. Many Eph receptors are frequently up-regulated in cancer cells and some are also present in the tumor microenvironment. Furthermore, modulating the activity of these receptors has been shown to affect tumor growth and metastatic spread. Several studies have demonstrated that disrupting the binding of Eph receptors to their ligands, the ephrins, in preclinical mouse tumor models results in decreased tumor growth, which is likely due at least in part to inhibition of tumor angiogenesis. Binding interactions between Eph receptors and their corresponding ephrin ligands, are promiscuous. Despite this lack of selectivity in the recognition of their natural ephrin ligands, high affinity ephrin-binding pockets of different Eph receptors show selectivity for artificial ligands such as peptides and small molecules, demonstrating that exquisitely specific targeting of individual Eph receptors can be achieved. Interestingly, upon binding to the Eph receptors, Eph-ligands get actively internalized (endocytosis) in the cell where they are directed into lysosomes.

A major drawback of conventional cancer drugs is the lack of selectivity in cancer cells, which results in undesirable cytotoxicity to normal cells. The potential therapeutic benefits that may be realized by targeting chemo-therapeutic agents directly to the Eph receptor sites has yet to be realized. The disclosure addresses these issues and further provides related advantages.

### SUMMARY OF THE DISCLOSURE

The disclosure provides compounds, compositions and methods for using peptide-Eph-ligands that are covalently linked to a variety of anti-cancer agents, for the selective delivery of these cytotoxic anti-cancer agents to cancer cells via the Eph receptors. The disclosed compounds, compounds and methods exploit the Eph-mediated internalization of chemotherapeutic drugs, thus providing an innovative and separate or complementary approach to the more conventional drug discovery efforts, and particularly targets cancer cells that over-express Eph receptors. The disclosed compounds include a delivery agent, a linker, and a chemotherapeutic agent that is covalently attached to the delivery agent via the linker. In some of the embodiments, the delivery agent is an Eph receptor-binding peptide or peptide derivative, and the chemotherapeutic drug includes but is not limited to taxol, doxorubicin, taxotere, campotechin, etoposide, and the like.

Thus, in one embodiment the disclosure provides a compound of Formula I:

EPH_T-L-D (I),

or a pharmaceutically acceptable salt, solvate or hydrate thereof, wherein:

EPH-T is an Eph receptor binding compound;

L is a linking group; and

D is a chemotherapeutic agent.

In another embodiment the disclosure provides a pharmaceutical composition comprising the compound of Formula I and a pharmaceutically acceptable solvent.

In another embodiment the disclosure provides a method of treating cancer, by administering a pharmacologically effective amount of a pharmaceutical composition comprising the compound of Formula I and a pharmaceutically acceptable solvent to a patient in need thereof.

In another embodiment the disclosure provides a method of targeting delivery of chemotherapeutic agents to the Eph receptor, the method comprising the steps of contacting the Eph receptor with the compound of Formula I.

In another embodiment the disclosure provides a method of preparing a compound of Formula I, by a) coupling the EPH_T (Eph receptor binding compound) to an alkynoic acid; b) coupling the D (chemotherapeutic agent) to an azide; and c) reacting the EPH-T (Eph receptor binding compound) coupled to an alkynoic acid with the D (chemotherapeutic agent) coupled to an azide in a 1,3-dipolar cycloaddition reaction to form a 1,4-disubstituted-1,2,3-triazole compound of Formula I.

### BRIEF DESCRIPTION OF THE DRAWINGS

Various features and advantages of the present disclosure will become more apparent by describing in detail exemplary embodiments thereof with reference to the attached drawings in which:

FIG. 1 illustrates YSA-taxol is more effective than taxol and doxorubicin inhibiting tumor growth in vivo without showing toxicity; and

FIG. 2 illustrates the measurements of lung metastases in PC3L xenograft mice treated with various agents.

### DETAILED DESCRIPTION OF THE DISCLOSURE

### Definitions

Abbreviations used herein have their conventional meaning within the chemical and biological arts. Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or cyclic hydrocarbon radical, or combinations thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, N-propyl, isopropyl, N-butyl, sec-butyl, tert-butyl, isobutyl, cyclobutyl, pentyl, cyclopentyl, hexyl, cyclohexyl, (cyclohexyl)methyl, cyclopropylmethyl, homologs and isomers of, for example, N-pentyl, N-hexyl, N-heptyl, N-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”.

The term “alkylene” by itself or as part of another substituent means a divalent radical derived from an alkyl, as exemplified, but not limited, by —CH₂CH₂CH₂CH₂—, —CH₂CH═CHCH₂—, —CH₂C═CCH₂—, —CH₂CH₂CH(CH₂CH₂CH₃)CH₂—. Typically, an alkyl (or alkylene) group will have from 1 to 24 carbon atoms. A “lower alkyl” or “lower alkylene” is a shorter chain alkyl or alkylene group, generally having eight or fewer carbon atoms.

As used herein, the terms “alkyl” and “alkylene” are interchangeable depending on the placement of the “alkyl” or “alkylene” group within the molecule.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, CH═CH—O—CH₃, —Si(CH₃)₃, —CH₂—CH═N—OCH₃, —CH═CH—N(CH₃)—CH₃, O—CH₃, —0—CH₂—CH₃ and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. As used herein, the terms “heteroalkyl” and “heteroalkylene” are interchangeable depending on the placement of the “heteroalkyl” or “heteroalkylene” group within the molecule.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, when the heteroatom is nitrogen, it can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively. As used herein, the terms “cycloalkyl” and “cycloalkylene” are interchangeable depending on the placement of the “cycloalkyl” or “cycloalkylene” group within the molecule. As used herein, the terms “heterocycloalkyl” and “heterocycloalkylene” are interchangeable depending on the placement of the “heterocycloalkyl” or “heterocycloalkylene” group within the molecule.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like. As used herein, the terms “haloalkyl” and “haloalkylene” are interchangeable depending on the placement of the “haloalkyl” or “haloalkylene” group within the molecule.

The term “aryl” means, unless otherwise stated, a polyunsaturated, aromatic, hydrocarbon substituent which can be a single ring or multiple rings, which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. For example, pyridine N-oxide moieties are included within the description of “heteroaryl.” A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, A-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, A-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent radicals of aryl and heteroaryl, respectively. As used herein, the terms “aryl” and “arylene” are interchangeable depending on the placement of the “aryl” and “arylene” group within the molecule. As used herein, the terms “heteroaryl” and “heteroarylene” are interchangeable depending on the placement of the “heteroaryl” and “heteroarylene” group within the molecule.

For brevity, the term “aryl” when used in combination with other terms (e.g., aryloxo, arylthioxo, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover aryls substituted with one or more halogens.

Where a heteroalkyl, heterocycloalkyl, or heteroaryl includes a specific number of members (e.g., “3 to 7 membered”), the term “member” referrers to a carbon or heteroatom.

The term “oxo” as used herein means an oxygen that is double bonded to a carbon atom.

Each of the above terms (e.g., “alkyl,” “heteroalkyl,” “cycloalkyl, and “heterocycloalkyl”, “aryl,” “heteroaryl” as well as their divalent radical derivatives) are meant to include both substituted and unsubstituted forms of the indicated radical. Substituents for each type of radical are provided below.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′—C(O)NR″R′″, —OC(O)NR′R″, —NR′C(O)R″, —NR′—C(O)NR″R′″, —NR′C(O)OR″, —NR′—C(NR″R′″)═NR″″, —S(O)R′, —S(O)₂R′, —S(O)₂NR′R″, —NR′SO₂R″, —CN and —NO₂ in a number ranging from zero to (2 m′+1), where m¹ is the total number of carbon atoms in such radical. R′, R″, R′″ and R″″ each independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the disclosure includes more than one R group, for example, each of the R groups is independently selected as are each R¹, R″, R′″ and R″″ groups when more than one of these groups is present. When R¹ and R″ are attached to the same nitrogen atom, they can be combined with the nitrogen atom to form a 4-, 5-, 6-, or 7-membered ring. For example, —NR¹R″ is meant to include, but not be limited to, 1-pyrrolidinyl and A-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl radicals above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR′C(O)R″, —NR′—C(O)NR″R′″, —NR′C(O)OR″, —NRC(NR′R″R′″)═NR″″, —NRC(NR′R″)═NR″″, —S(O)R′, —S(O)₂R′, —S(O)₂NR′R″, —NR′SO₂R″, —CN and —NO₂, in a number ranging from zero to the total number of open valences on aromatic ring system; and where R′, R″, R′″ and R″″ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from O to 3. Alternatively, two of the substituents on adjacent atoms of aryl or heteroaryl ring may optionally be replaced with a substituent of the formula -A-(CH₂)ᵣ—B—, wherein A and B are independently —CR′R″—, —O—, —NR′—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of aryl or heteroaryl ring may optionally be replaced with a substituent of the formula —(CR′R″)ₛ—X′—(C″R′″)_{d}—, where s and d are independently integers of from O to 3, and X′ is —O—, —NR′—, —S—, —S(O)—, —S(O)₂—, or —S(O)₂NR′—. The substituents R′, R″, and R′″ are independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

An “aminoalkyl” as used herein refers to an amino group covalently bound to an alkylene linker. The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

A “substituent group,” as used herein, means a group selected from at least the following moieties: (A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and (B) alkyl, heteroalkyl, cycloalkyl, heterocycloalkyl, aryl, and heteroaryl, substituted with at least one substituent selected from: (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, and heteroaryl, substituted with at least one substituent selected from: (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The neutral forms of the compounds are regenerated by contacting the salt with a base or acid and isolating the parent compound in the conventional manner. The parent form of the compound differs from the various salt forms in certain physical properties, such as solubility in polar solvents.

Certain compounds of the disclosure can exist in unsolvated forms as well as solvated forms, including hydrated forms. In general, the solvated forms are equivalent to unsolvated forms and are encompassed within the scope of the disclosure. Certain compounds of the disclosure may exist in multiple crystalline or amorphous forms. In general, all physical forms are equivalent for the uses contemplated by the disclosure.

Certain compounds of the disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the disclosure. The compounds of the disclosure do not include those which are known in art to be too unstable to synthesize and/or isolate. The disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the disclosure.

The compounds of the disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the disclosure, whether radioactive or not, are encompassed within the scope of the disclosure.

The term “pharmaceutically acceptable salts” is meant to include salts of active compounds which are prepared with relatively nontoxic acids or bases, depending on the particular substituent moieties found on the compounds described herein. When compounds of the disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, mono-hydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science, 66:1-19 (1977)). Certain specific compounds of the disclosure contain both basic and acidic functionalities that allow the compounds to be converted into either base or acid addition salts.

In addition to salt forms, the disclosure provides compounds, which are in a prodrug form. Prodrugs of the compounds described herein are those compounds that readily undergo chemical changes under physiological conditions to provide the compounds of the disclosure. Additionally, prodrugs can be converted to the compounds of the disclosure by chemical or biochemical methods in an ex vivo environment. For example, prodrugs can be slowly converted to the compounds of the disclosure when placed in a transdermal patch reservoir with a suitable enzyme or chemical reagent.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

Description of compounds of the disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” in reference to a particular disease includes prevention of the disease.

The symbol >˜w- denotes the point of attachment of a moiety to the remainder of the molecule.

The term “Eph-receptor” refers to the Ephrin receptor family.

The term “YSA polypeptide analog” includes any polypeptide having an amino acid residue sequence substantially the same as the YSA polypeptide in which one or more residues have been conservatively substituted with a functionally similar residue and which has the ability to bind to Eph receptors. Conservative substitutions of encoded amino acids include, for example, amino acids that belong within the following groups: (1) non-polar amino acids (Gly, Ala, Val, Leu, and He); (2) polar neutral amino acids (Cys, Met, Ser, Thr, Asn, and Gin); (3) polar acidic amino acids (Asp and Glu); (4) polar basic amino acids (Lys, Arg and H is); and (5) aromatic amino acids (Phe, Tip, Tyr, and His. Other groupings of amino acids can be found, for example in Taylor, J. Theor. Biol. 119:205-218 (1986), which is incorporated herein by reference. Other minor modifications include polypeptides so long as the polypeptide retains some or all of its function as YSA polypeptide. Un-natural, structurally similar amino acid derivatives are also included within the meaning of conservative substitution.

The term “effective amount” of a compound refers a non-toxic but sufficient amount of the compound that provides a desired effect. This amount may vary from subject to subject, depending on the species, age, and physical condition of the subject, the severity of the disease that is being treated, the particular compound used, its mode of administration, and the like. A suitable effective amount may be determined by one of ordinary skill in the art. For example, the disclosed compounds can be administered at a concentration of about 0.1-50 mg/kg, in certain aspects between 0.1 and 5 mg/kg. In some aspects of the disclosure, an effective amount is at least 0.5 mg/kg, for example, 0.5 mg/kg to about 10 mg/kg, or 0.5 mg/kg to about 5 mg/kg. In certain aspects of the disclosure, the disclosed compounds can be administered at a concentration of about 0.5 mg/kg or 1 mg/kg.

The term “pharmaceutically acceptable” refers to a compound, additive or composition that is not biologically or otherwise undesirable. For example, the additive or composition may be administered to a subject along with a compound of the disclosure without causing any undesirable biological effects or interacting in an undesirable manner with any of the other components of the pharmaceutical composition in which it is contained.

As used herein, the term “patient” refers to organisms to be treated by the methods of the present disclosure. Such organisms include, but are not limited to, humans. In the context of the disclosure, the term “subject” generally refers to an individual who will receive or who has received treatment for the treatment of a disease, disorder or pathology.

### Targeted Delivery of the Eph Receptor (EPH_T)

The disclosure provides compounds comprising a delivery agent targeting the Eph receptor (EPH_T), linked to a chemotherapeutic agent having Formula I:

EPH_T-L-D (I),

wherein EPH_T is an Eph receptor binding compound, for example, a peptide or peptide mimetic; L is a linking group; and D is a chemotherapeutic agent. A variety of Eph-targeting molecules can be synthesized by appropriately coupling the YSA peptide, via a linker, with conventional chemotherapeutic agents including taxol, doxorubicin, taxotere, etoposide and campotechin The resulting compounds are actively transported inside the Eph-overexpressing cancer cells thereby allowing for targeted delivery of the drugs to the tumor. In an example described herein, a taxol derivative possesses an in vivo activity in mice models of prostate cancer, which is remarkably superior to the activity of the unconjugated parent drug given at the same molar concentration.

Eph receptor tyrosine kinases represent promising disease targets because they are differentially expressed in pathological versus normal tissues. For example, the EphA2 receptor is up-regulated in transformed cells and tumor vasculature where it likely contributes to cancer pathogenesis. An ephrin mimetic peptide that selectively targets the EphA2 receptor is the YSA peptide. The YSA peptide targets the ligand binding domain of EphA2 and competes with the ephrin ligands for binding (Koolpe, M. et al. The Journal of Biological Chemistry, Vol. 277, No. 49, pp 46974-79).

The YSA peptide has the following structure:

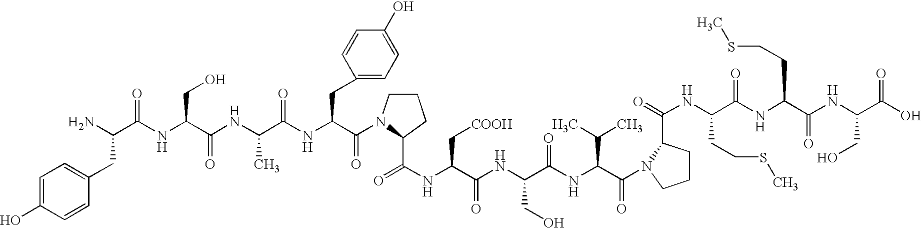

### YSA Peptide,

which may be abbreviated as “YSAYPDSVPMMS,” using the single letter code abbreviation for amino acids as shown below:

### Nonpolar Amino Acids (Hydrophobic)

| | |
|---|---|
| glycine | G |
| alanine | A |
| valine | V |
| leucine | L |
| isoleucine | I |
| methionine | M |
| phenylalanine | F |
| tryptophan | W |
| proline | P |

### Polar Amino Acids (Hydrophilic)

| | |
|---|---|
| serine | S |
| threonine | T |
| cysteine | C |
| tyrosine | Y |
| asparagine | N |
| glutamine | Q |

### Electrically Charged Amino Acids (Negative)

| | |
|---|---|
| aspartic acid | D |
| glutamic acid | E |

### Electrically Charged Amino Acids (Positive)

| | |
|---|---|
| lysine | K |
| arginine | R |
| histidine | H |

The above YSA peptide can be replaced by other amino acid sequences including not-naturally occurring amino acids. Examples of such motifs include but are not limited to a YSA polypeptide having the amino acid sequence of YSAYPDSVPMMS or a YSA polypeptide analog thereof YSA polypeptide analogs refer to any polypeptide and polypeptide mimetic having an amino acid sequence substantially the same as the YSA polypeptide with one or more conservative substitutions. Substitution with unnatural amino acid residues is also included within the meaning of conservative substitution. In one example, the YSA motif has the amino sequence of YSAYPDSVPnLnLS, wherein nL stands for nor-Leucine. Other conservative substitutions for amino acids include any of the interchangeable amino acids listed for the nonpolar amino acids (hydrophobic), polar amino acids (hydrophilic), electrically charged amino acids (negative), and the electrically charged amino acids (positive), respectively.

Click chemistry is an approach to the synthesis of drug-like molecules that can accelerate the drug discovery process by utilizing a few practical and reliable reactions. In 2001, Sharpless and coworkers defined what makes a click reaction as one that is wide in scope and easy to perform, uses only readily available reagents, and is insensitive to oxygen and water. In fact, in several instances water is the ideal reaction solvent, providing the best yields and highest rates. Reaction work-up and purification uses benign solvents and avoids chromatography. Of the reactions comprising the click universe, the “perfect” example is the 1,3-dipolar cycloaddition of alkynes to azides to form 1,4-disubstituted-1,2,3-triazoles (see, Scheme 1).

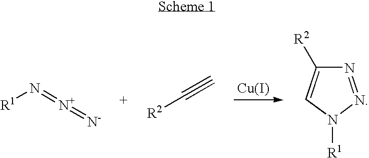

The copper(I)-catalyzed reaction is mild and very efficient, requiring no protecting groups, and requiring no purification in many cases. The azide and alkyne functional groups are largely inert towards biological molecules and aqueous environments, which allows the use of the 1,3-dipolar cycloaddition in target guided synthesis and activity-based protein profiling. The triazole has similarities to the ubiquitous amide moiety found in nature, but unlike amides, is not susceptible to cleavage. Additionally, they are nearly impossible to oxidize or reduce. Using Cu(II) salts with ascorbate has been the method of choice for preparative synthesis of 1,2,3-triazoles, but is problematic in biocojugation applications. However, tris[(1-benzyl-1H-1,2,3-triazol-4-yl)methyl]amine, TBTA, has been shown to effectively enhance the copper-catalyzed cycloaddition without damaging biological scaffolds.

Thus, in one aspect the disclosure provides a compound of Formula I:

EPH_T-L-D (I),

or a pharmaceutically acceptable salt, solvate or hydrate thereof, wherein:

EPH-T is an Eph receptor binding compound;

L is a linking group; and

D is a chemotherapeutic agent.

In another aspect the disclosure provides a compound of Formula I, wherein the EPH-T is a YSA peptide having Formula II:

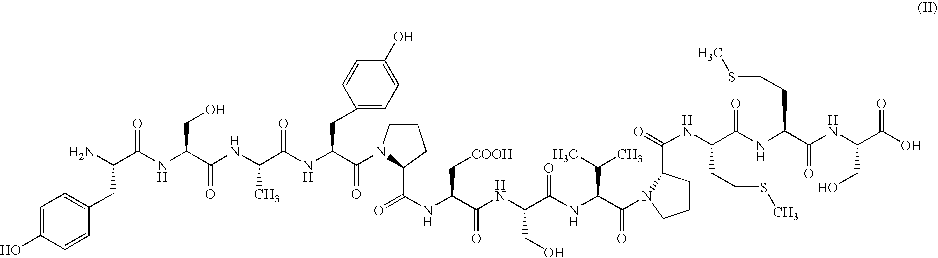

or amino acid sequence YSAYPDSVPMMS, wherein Y is tyrosine; S is serine; A is alanine; P is proline; D is aspartic acid; V is valine; and M is methionine.

In another aspect the disclosure provides a compound of Formula I, wherein the EPH-T is a YSA peptide having Formula II, wherein the YSA peptide having Formula II or amino acid sequence YSAYPDSVPMMS, is substituted with any of the amino acid substitutions as follows:

each Y is optionally S, T, C, N or Q;

each S is optionally T, C, Y, N or Q;

each A is optionally G, V, L, nL, I, M, F, W or P;

each P is optionally G, A, V, L, nL, I, M, F or W;

each D is optionally E;

each V is optionally G, A, L, nL, I, M, F, W or P; and

each M is optionally G, A, V, L, nL, I, F, W or P,

wherein: G is glycine; A is alanine; V is valine; L is leucine; nL is nor-Leucine; I is isoleucine; M is methionine; F is phenylalanine; W is tryptophan; P is proline; S is serine; T is threonine; C is cysteine; Y is tyrosine; N is asparagine; Q is glutamine; D is aspartic acid; E is glutamic acid; K is lysine; R is arginine; and H is histidine.

In another aspect the disclosure provides a compound of Formula I, wherein the EPH-T is a YSA peptide having Formula II, wherein the YSA peptide having Formula II or amino acid sequence YSAYPDSVPMMS, has amino acid sequence: YSAYPDSVPnLnLS, wherein nL is nor-Leucine.

In another aspect the disclosure provides a compound of Formula I, wherein the linking group has Formula III or IV:

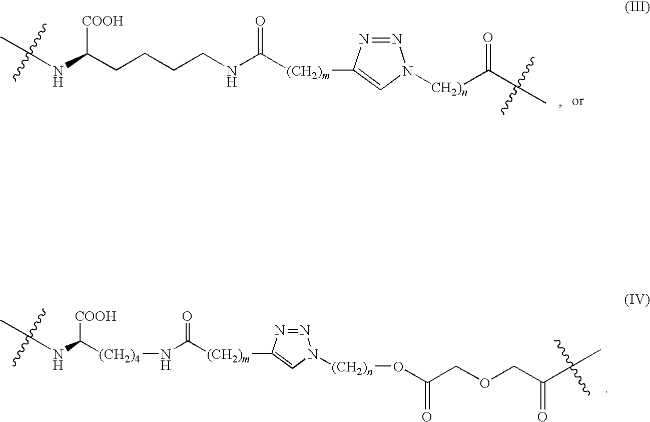

wherein m and n are each independently integers from 0, 1, 2, 3, 4, 5, 6, 7, 8, 9, 10, 11, or 12.

In another aspect the disclosure provides a compound of Formula I, wherein the linking group has Formula III or IV, wherein m is 2; and n is 2.

In another aspect the disclosure provides a compound of Formula I, wherein the chemotherapeutic agent is taxol, doxorubicin, taxotere, campotechin, or etoposide.

In another aspect the disclosure provides a compound of Formula I, wherein the compound of Formula I has Formula V:

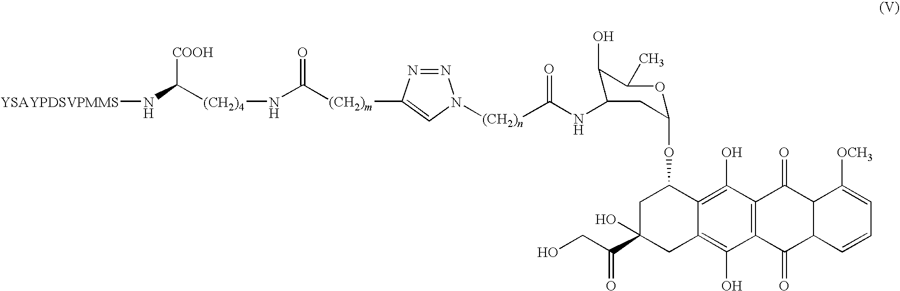

wherein m and n are each independently an integer from 0, 1, 2, 3, 4, 5, 6, 7, 8, 9, 10, 11, or 12.

In another aspect the disclosure provides a compound of Formula I, wherein the compound of Formula I has Formula VI:

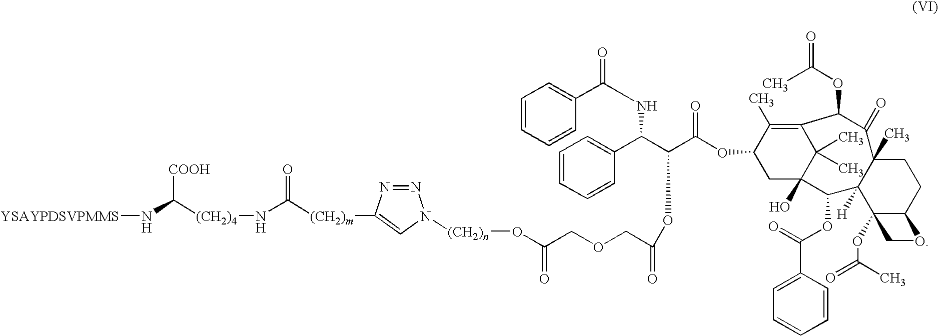

In another aspect the disclosure provides a compound of Formula I, wherein the compound of Formula I has Formula VII:

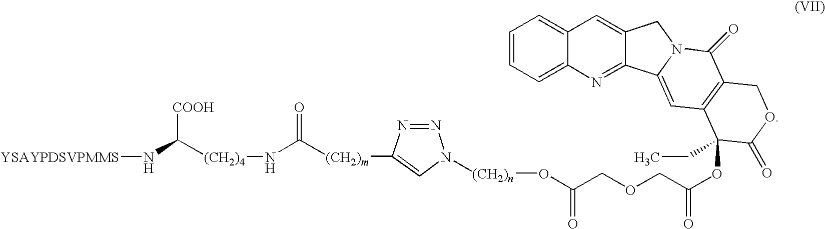

In another aspect the disclosure provides a compound of Formula VIII:

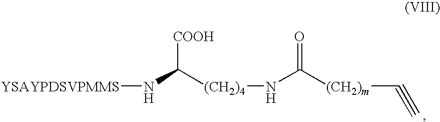

or a pharmaceutically acceptable salt, solvate or hydrate thereof, wherein m is independently an integer from 0, 1, 2, 3, 4, 5, 6, 7, 8, 9, 10, 11, or 12.

In another aspect the disclosure provides a compound of Formula IX:

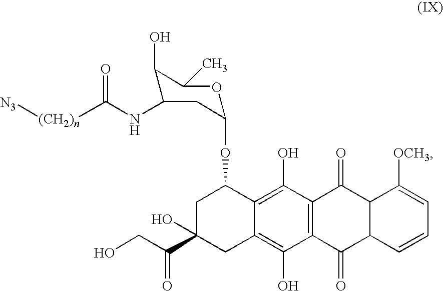

or a pharmaceutically acceptable salt, solvate or hydrate thereof, wherein n is independently an integer from 0, 1, 2, 3, 4, 5, 6, 7, 8, 9, 10, 11, or 12.

In another aspect the disclosure provides a compound of formula X:

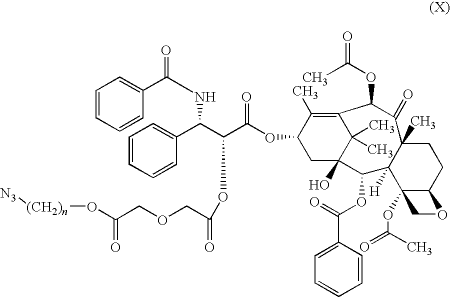

or a pharmaceutically acceptable salt, solvate or hydrate thereof, wherein n is independently an integer from 0, 1, 2, 3, 4, 5, 6, 7, 8, 9, 10, 11, or 12.

In another aspect the disclosure provides a compound of formula XI:

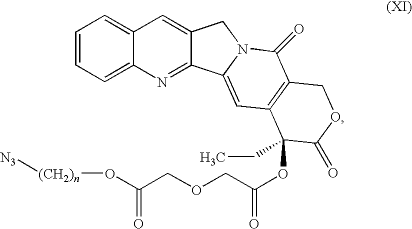

or a pharmaceutically acceptable salt, solvate or hydrate thereof, wherein n is independently an integer from 0, 1, 2, 3, 4, 5, 6, 7, 8, 9, 10, 11, or 12.

In another aspect the disclosure provides a pharmaceutical composition including the compound of Formula I and a pharmaceutically acceptable solvent.

In another aspect the disclosure provides a method of treating cancer by administering a pharmacologically effective amount of a pharmaceutical composition, including the compound of Formula I and a pharmaceutically acceptable solvent, to a patient in need thereof.

In another aspect the disclosure provides a method of targeting delivery of chemotherapeutic agents to the Eph receptor, the method comprising the steps of contacting the Eph receptor with a compound of Formula I.

In another aspect the disclosure provides a method of preparing a compound of Formula I: EPH_T-L-D (I), by: a) coupling the EPH_T (Eph receptor binding compound) to an alkynoic acid; b) coupling the D (chemotherapeutic agent) to an azide; and c) reacting the EPH-T (Eph receptor binding compound) coupled to an alkynoic acid with the D (chemotherapeutic agent) coupled to an azide in a 1,3-dipolar cycloaddition reaction to form a 1,4-disubstituted-1,2,3-triazole compound of Formula I.

In another aspect the disclosure provides a method of preparing a compound of Formula I, wherein the EPH-T is a YSA peptide having Formula II:

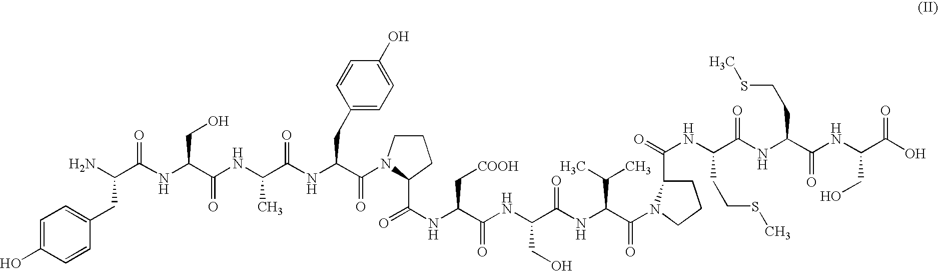

or amino acid sequence YSAYPDSVPMMS, wherein Y is tyrosine; S is serine; A is alanine; P is proline; D is aspartic acid; V is valine; and M is methionine.

In another aspect the disclosure provides a method of preparing a compound of Formula I, wherein the YSA peptide having Formula II or amino acid sequence YSAYPDSVPMMS, is substituted with any of the amino acid substitutions as follows: each Y is optionally S, T, C, N or Q; each S is optionally T, C, Y, N or Q; each A is optionally G, V, L, nL, I, M, F, W or P; each P is optionally G, A, V, L, nL, I, M, F or W; each D is optionally E; each V is optionally G, A, L, nL, I, M, F, W or P; and each M is optionally G, A, V, L, nL, I, F, W or P, wherein: G is glycine; A is alanine; V is valine; L is leucine; nL is nor-Leucine; I is isoleucine; M is methionine; F is phenylalanine; W is tryptophan; P is proline; S is serine; T is threonine; C is cysteine; Y is tyrosine; N is asparagine; Q is glutamine; D is aspartic acid; E is glutamic acid; K is lysine; R is arginine; and H is histidine.

In another aspect the disclosure provides a method of preparing a compound of Formula I, wherein the YSA peptide having Formula II or amino acid sequence YSAYPDSVPMMS, has amino acid sequence: YSAYPDSVPnLnLS, wherein nL is nor-Leucine.

In another aspect the disclosure provides a method of preparing a compound of Formula I, wherein the 1,4-disubstituted-1,2,3-triazole compound has Formula III or IV:

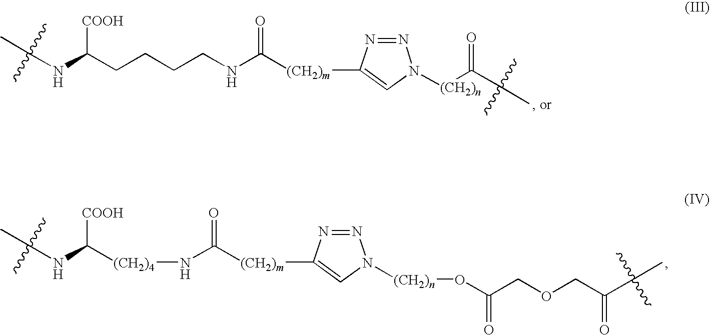

wherein m and n are each independently integers from 0, 1, 2, 3, 4, 5, 6, 7, 8, 9, 10, 11, or 12.

In another aspect the disclosure provides a method of preparing a compound of Formula I, wherein the 1,4-disubstituted-1,2,3-triazole compound has Formula III or IV, wherein m is 2; and n is 2.

In another aspect the disclosure provides a method of preparing a compound of Formula I, wherein the chemotherapeutic agent is taxol, doxorubicin, taxotere, campotechin, or etoposide.

In another aspect the disclosure provides a method of preparing a compound of Formula I, wherein the compound of Formula I has Formula V:

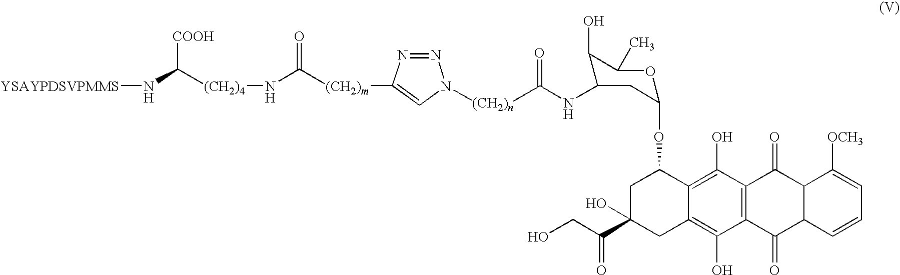

wherein m and n are each independently an integer from 0, 1, 2, 3, 4, 5, 6, 7, 8, 9, 10, 11, or 12.

In another aspect the disclosure provides a method of preparing a compound of Formula I, wherein the compound of Formula I has Formula VI:

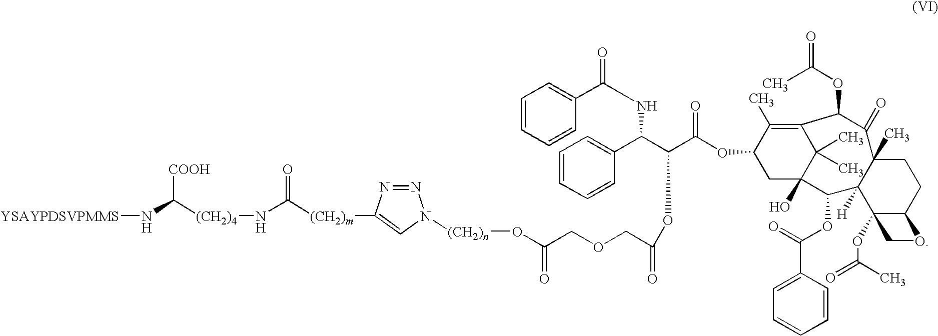

In another aspect the disclosure provides a method of preparing a compound of Formula I, wherein the compound of Formula I has Formula VII:

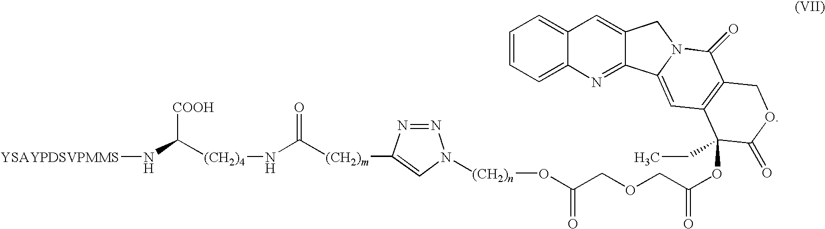

In another aspect the disclosure provides a method of preparing a compound of Formula I, wherein the coupling of the EPH_T (Eph receptor binding compound) to an alkynoic acid provides a compound of Formula VIII:

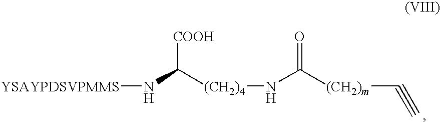

or a pharmaceutically acceptable salt, solvate or hydrate thereof, wherein m is independently an integer from 0, 1, 2, 3, 4, 5, 6, 7, 8, 9, 10, 11, or 12.

In another aspect the disclosure provides a method of preparing a compound of Formula I, wherein the coupling of the D (chemotherapeutic agent) to an azide provides a compound of Formula IX:

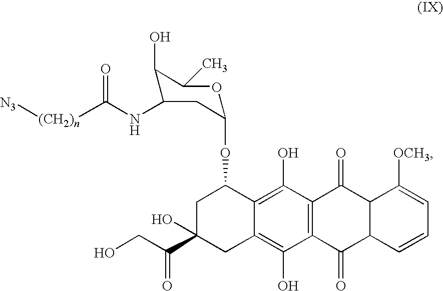

or a pharmaceutically acceptable salt, solvate or hydrate thereof, wherein n is independently an integer from 0, 1, 2, 3, 4, 5, 6, 7, 8, 9, 10, 11, or 12.

In another aspect the disclosure provides a method of preparing a compound of Formula I, wherein the coupling of the D (chemotherapeutic agent) to an azide provides a compound of formula X:

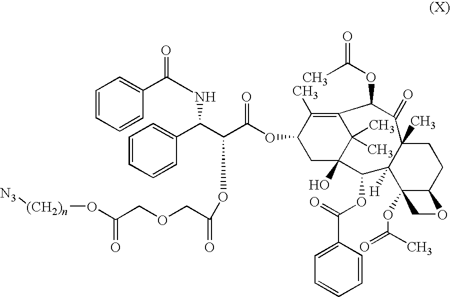

or a pharmaceutically acceptable salt, solvate or hydrate thereof, wherein n is independently an integer from 0, 1, 2, 3, 4, 5, 6, 7, 8, 9, 10, 11, or 12.

In another aspect the disclosure provides a method of preparing a compound of Formula I, wherein the coupling of the D (chemotherapeutic agent) to an azide provides a compound of formula XI:

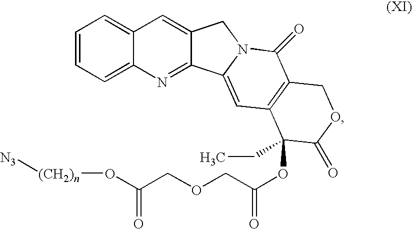

or a pharmaceutically acceptable salt, solvate or hydrate thereof, wherein n is independently an integer from 0, 1, 2, 3, 4, 5, 6, 7, 8, 9, 10, 11, or 12.

The compounds of the disclosure are capable of inhibiting tumor growth and metastases and may, therefore, be used for the treatment of cancer. Accordingly, the disclosed compounds or pharmaceutically acceptable salts thereof can be used for preparing pharmaceutical compositions, e.g., by combining these compounds and pharmaceutically acceptable carriers. The pharmaceutical compositions can then be used in pharmacologically effective doses as anti-cancer agents.

Various synthetic schemes can be designed for manufacturing the disclosed compounds. Synthetic processes for these compounds are reported in the examples.

Pharmaceutically acceptable salts of the compounds of the disclosure may be obtained using standard procedures well known in the art, for example by causing a reaction between a sufficiently basic compound such as an amine and a suitable acid affording a physiologically acceptable anion. Alkali metal (for example, sodium, potassium or lithium) or alkaline earth metal (for example calcium) salts of carboxylic acids can also be made.

The disclosed compounds can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

The active compound may also be administered intravenously or intraperitoncally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The disclosed compounds may also be administered in an amount of between about 0.01 and 25 mg/kg body weight. In certain aspects, the compounds can be administered at a concentration equal to or greater than 1 mg/kg, for example between about 3 and about 20 mg/kg. In other aspects, the disclosed compounds can be is administered at a concentration of between about 5 and about 15 mg/kg. In other aspects, the disclosed compounds can be administered at between about 7 and about 12 mg/kg, for example at 9 mg/kg. It will be understood that the disclosure provides a basis for further studies in humans to more precisely determine effective amounts in humans. Doses used for rodent studies provide a basis for the ranges of doses indicated herein for humans and other mammals.

The route of delivery of the compounds employed by disclosed methods may be determined by the particular disorder. The compounds may be delivered orally, intravenously, intraperitoneally, intramuscularly, subcutaneously, intranasally, and intradermally, as well as, by transdermal delivery (e.g., with a lipid-soluble carrier in a skin patch placed on skin), or even by gastrointestinal delivery (e.g., with a capsule or tablet). Furthermore, the compounds used in the methods of the disclosure, in certain aspects are delivered directly to the brain or certain regions of the brain to activate or inhibit receptors at specific brain sites producing the desirable effect without inhibiting or activating receptors at other brain sites, thus avoiding undesirable side-effects or actions that may counteract the beneficial therapeutic action mediated by the former site(s). The dosage will be sufficient to provide an effective amount of a compound either singly or in combination, as discussed above. Some variation in dosage will necessarily occur depending upon the condition of the patient being treated, and the physician will, in any event, determine the appropriate dose for the individual patient. The dose will depend, among other things, on the body weight, physiology, and chosen administration regimen.

The compounds employed in disclosed methods can be administered alone or in combination with pharmaceutically acceptable carriers, in either single or multiple doses. Suitable pharmaceutical carriers include inert solid diluents or fillers, sterile aqueous solutions, and various nontoxic organic solvents. The pharmaceutical compositions formed by combining one or more compounds with the pharmaceutically acceptable carrier are then readily administered in a variety of dosage forms such as tablets, lozenges, syrups, injectable solutions, and the like. These pharmaceutical carriers can, if desired, contain additional ingredients such as flavorings, binders, excipients, and the like. Thus, for purposes of oral administration, tablets containing various excipients such as sodium citrate, calcium carbonate, and calcium phosphate are employed along with various disintegrants such as starch, and potato or tapioca starch, alginic acid, and certain complex silicates, together with binding agents such as polyvinylpyrolidone, sucrose, gelatin, and acacia. Additionally, lubricating agents, such as magnesium stearate, sodium lauryl sulfate, and talc are often useful for tableting purposes. Solid compositions of a similar type may also be employed as fillers in salt and hard-filled gelatin capsules. Appropriate materials for this purpose include lactose or milk sugar and high molecular weight polyethylene glycols.

The disclosed compounds or one of its pharmaceutically acceptable salts as defined herein, may be useful in the treatment of cancer and manufacturing of a pharmaceutical composition intended for the treatment of cancers, whatever their nature and their degree of anaplasia, in particular including cancers such as melanomas, carcinomas, sarcomas, fibrosarcomas, leukaemias, lymphomas, neuroblastomas, medulloblastomas, glioblsatomas, astrocytomas, angioblastomas, meningiomas, retinoblastomas, prolactinomas, macrobulimia, leiomyosarcomas, mesotheliomas, choriocarcinomas, pheochromocytomas, myelomas, polycythemias, angiosarcomas, extra-skeletal chondrosarcomas, hemangiosarcomas, osteosarcomas, and chondrosarcomas.

By way of example of such cancers, the following can be cited: pancreatic cancer, cancers of the oropharynx, stomach cancer, cancer of the oesophagus, colon and rectal cancer, brain cancer, in particular gliomas, ovarian cancer, liver cancer, kidney cancer, cancer of the larynx, thyroid cancer, lung cancer, bone cancer, multiple myelomas, mesotheliomas and melanomas, skin cancer, breast cancer, prostate cancer, bladder cancer, cancer of the uterus, testicular cancer, non-Hodgkin's lymphoma, leukaemia, Hodgkin's disease, cancer of the tongue, cancer of the duodenum, bronchial cancer, pancreatic cancer and soft tissue cancers, as well as metastatic secondary locations of the aforementioned cancers, such as in the lung, liver and breast.

The disclosed compounds and compositions may be used in combination with one or more chemotherapeutic agents including but not limited to methotrexate, cisplatin/carboplatin; canbusil; dactinomycin; taxol (paclitaxol), antifolate, colchicine, demecolcine, etoposide, taxane/taxol, docetaxel, doxorubicin, anthracycline antibiotic, doxorubicin, daunorubicin, caminomycin, epirubicin, idarubicin, mitoxanthrone, 4-demethoxy-daunomycin, 11-deoxydaunorubicin, 13-deoxydaunorubicin, adriamycin-14-benzoate, adriamycin-14-octanoate, adriamycin-14-naphthaleneacetate, trastuzumab, bevacizumab, OSI-774, or Vitaxin.

When aqueous suspensions of elixirs are desired for oral administration, the compounds may be combined with various sweetening or flavoring agents, colored matter or dyes, and if desired, emulsifying or suspending agents, together with diluents such as water, ethanol, propylene glycol, glycerin, and combinations thereof. For parenteral administration, solutions of preparation in sesame or peanut oil or in aqueous polypropylene glycol are employed, as well as sterile aqueous saline solutions of the corresponding water soluble pharmaceutically acceptable metal salts previously described. Such an aqueous solution should be suitably buffered if necessary and the liquid diluent first rendered isotonic with sufficient saline or glucose. These particular aqueous solutions are especially suitable for intravenous, intramuscular, subcutaneous, and intraperitoneal injection. The sterile aqueous media employed are all readily obtainable by standard techniques well known to those skilled in the art.

The pharmaceutical dosage forms suitable for injection or infusion can include sterile aqueous solutions or dispersions or sterile powders comprising the active ingredient which arc adapted for the extemporaneous preparation of sterile injectable or infusible solutions or dispersions, optionally encapsulated in liposomes. In many cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions arc prepared by incorporating the active compound in the required amount in the appropriate solvent with various of the other ingredients enumerated above, as required, followed by filter sterilization. In the case of sterile powders for the preparation of sterile injectable solutions, the methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used Lo impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the disclosed compounds can be determined by comparing their in vitro activity, and in vivo activity in animal models. Methods for the extrapolation of effective dosages in mice, and other animals, to humans are known to those having ordinary skill in the art who can, for example, be guided by the procedures described in the art, for example as described in U.S. Pat. No. 4,938,949.

Generally, the concentration of the disclosed compounds in a liquid composition, such as a lotion, can be between about 0.1 and 25 mass %, such as between about 0.5 and 10 mass %. The concentration in a semi-solid or solid composition such as a gel or a powder can be between about 0.1 and 25 mass %, such as between about 0.5 and 2.5 mass %.

The amount of the disclosed compounds or an active salt or derivative thereof, required for use in treatment will vary with the particular salt selected, the route of administration, the nature of the condition being treated and the age and condition of the patient, ultimately, with the discretion of the attendant physician or clinician.

### EXAMPLES

For further illustration of various aspects of the present disclosure, several specific examples will now be described. It should be understood however that these examples are for illustrative purposes only, and are not intended to limit the scope of the present disclosure.

### Example 1

### Synthesis of YSA-Doxorubicin (YSA-DOX)

Doxorubicin is an anthracycline natural product that is frequently used to treat breast cancer, liver cancer, soft-tissue sarcomas, and non-Hodgkin's lymphoma. Doxorubicin kills tumor cells by causing topoisomerase II-stimulated DNA strand breaks and potentially other mechanisms. Like other cytotoxic drugs, doxorubicin doses are limited by unwanted toxicity to non-tumor tissues. Myelosuppression is the dose-limiting toxicity for doxorubicin, although stomatitis, mucositis, and alopecia are also frequently observed. In addition to these typical chemotherapeutic toxicities, doxorubicin causes cardiomyopathy that depends on the cumulative dose of doxorubicin. Increasing the therapeutic index of doxorubicin could benefit cancer treatment by allowing greater concentrations of doxorubicin in tumors and thereby potentially greater tumor growth inhibition. Alternatively, increasing the therapeutic index of doxorubicin could allow reduced toxicities and thereby permit the use of other cytotoxic agents that would be prevented because of overlapping toxicities. Hence, the therapeutic index of doxorubicin may be improved by creating YSA-doxorubicin derivatives that selectively target the tumor environment. Described below is the coupling of doxorubicin to the YSA peptide, which is subsequently coupled via click chemistry to YSA-alkynes as described earlier. Substitution of the amino group of doxorubicin has been reported in the literature and the resulting compounds do not generally lose the cytotoxic properties of the parent compound. In fact, by using a similar strategy, doxorubicin-albumin conjugates containing a MMP-2 specific cleavage site have been reported as very effective yet less toxic than doxorubicin alone. The described approach leads to tumor specific YSA-doxorubicin compounds with markedly improved therapeutic index than the original drug.

The synthesis of YSA-doxorubicin (YSA-DOX) is outlined below.

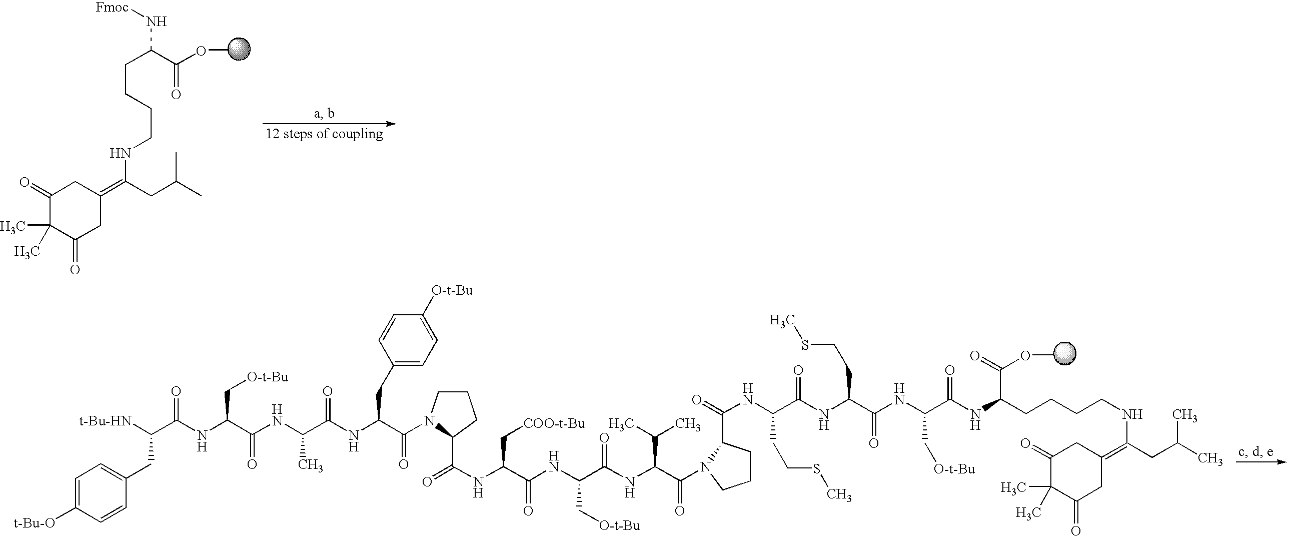

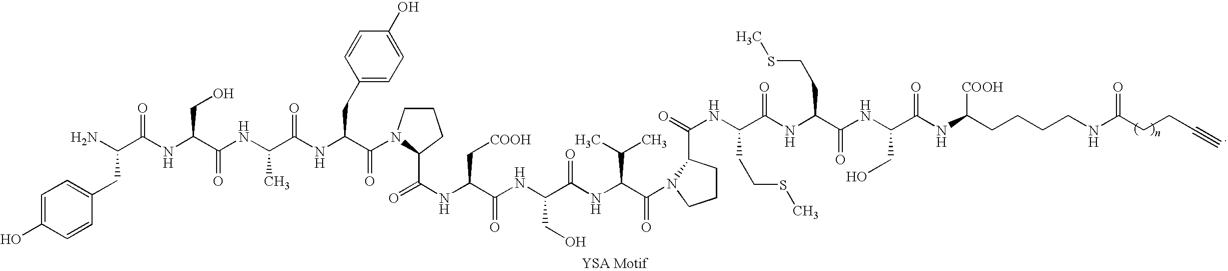

a) 20% piperidine, DMF, rt, 2 h; b) Fmoc amino acid or Boc amino acid, HTBU/HOBt/DIEPA, rt, 6 h; c) NH₂NH₂, DMF, rt, 30 min; d) 5-hexynoic acid, HTBU, HOBt, DIEPA, rt, 6 h; e) TFA, phenol, thioanisole, EDT, rt, 3 h.

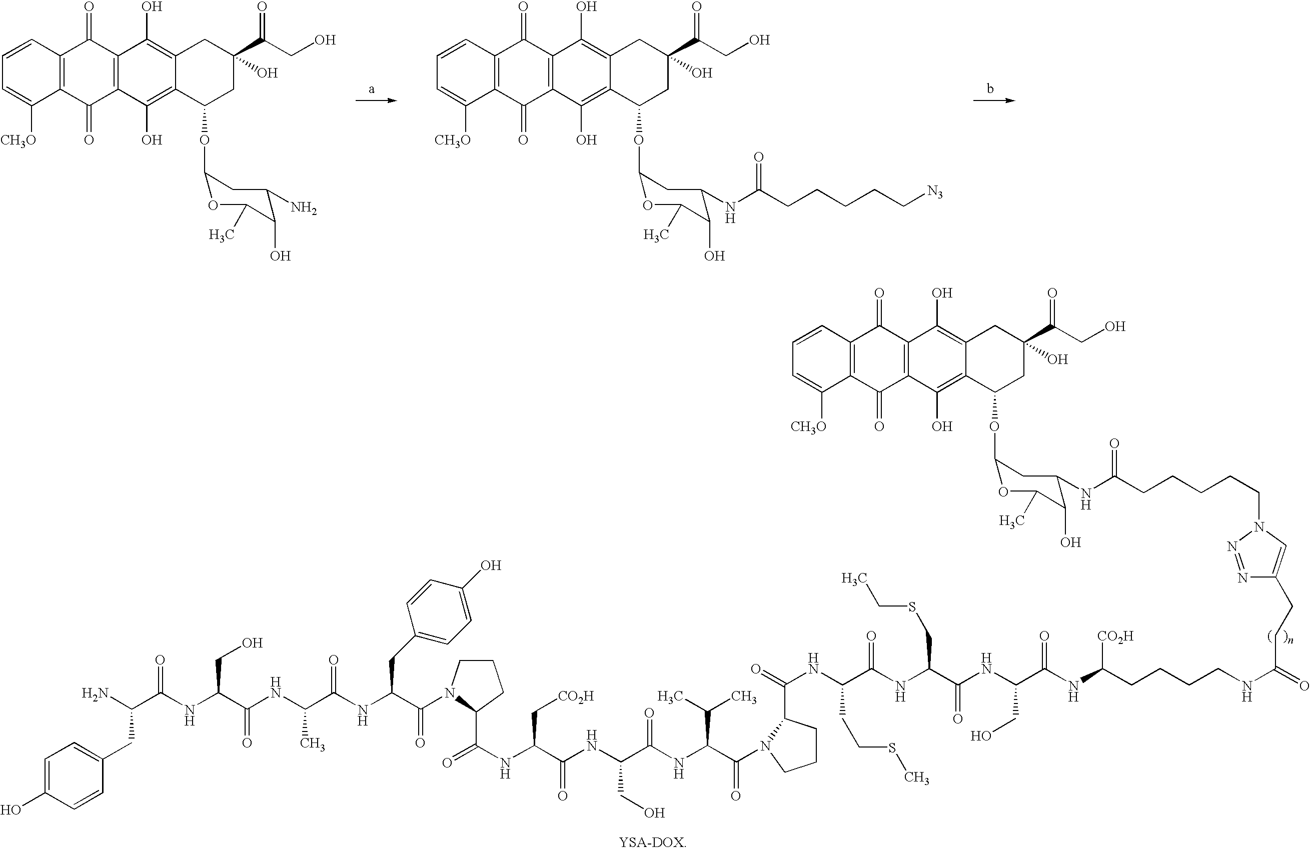

a) HBTU, DIPEA, DMF, 6-azido hexanoic acid; rt, 12 h, 81%; b) CuS0₄, sodium ascorbate, MeOH, YSA motif, rt, 2 d, 65%.

### Example 2

### Synthesis of YSA Taxol (YSA-TAX)

Solvent-based delivery vehicles for chemotherapy agents have been instrumental in providing a means for hydrophobic agents to be administered intravenously. These solvents, however, have been associated with serious and dose-limiting toxicities. Solvent-based formulations of taxanes, a highly active class of cytotoxic agents, are associated with hypersensitivity reactions, neutropenia, and neuropathy. Nanoparticle technology utilizing the human protein albumin exploits natural pathways to selectively deliver larger amounts of drug to tumors while avoiding some of the toxicities of solvent-based formulations. 130 nM albumin-bound paclitaxel (nab-paclitaxel; Abraxane®) was recently approved for use in patients with metastatic breast cancer who have failed combination therapy. In a randomized, phase III study in metastatic breast cancer, nab-paclitaxel was found to have improved efficacy and safety compared with conventional, solvent-based paclitaxel. Preliminary data also suggest roles for nab-paclitaxel as a single agent and in combination therapy for first-line treatment of metastatic breast cancer as well as in other solid tumors, including non-small-cell lung cancer, ovarian cancer, and malignant melanoma. Clinical trials are underway using formulations of other water-insoluble anticancer agents, such as docetaxel and rapamycin. Based on these studies and our preliminary work with the EphA2 receptor and the YSA peptide, a series of YSA-taxol derivatives were prepared.

The synthetic scheme adopted follows that of the previously reported derivatives used for the preparation of Abraxane®, which follows well established chemistry. Hence, a variety of azide-linkers can be introduced in taxol and the above described method can be used to couple the resulting compounds with the previously prepared YSA-alkyne derivatives. This route is schematically summarized below.

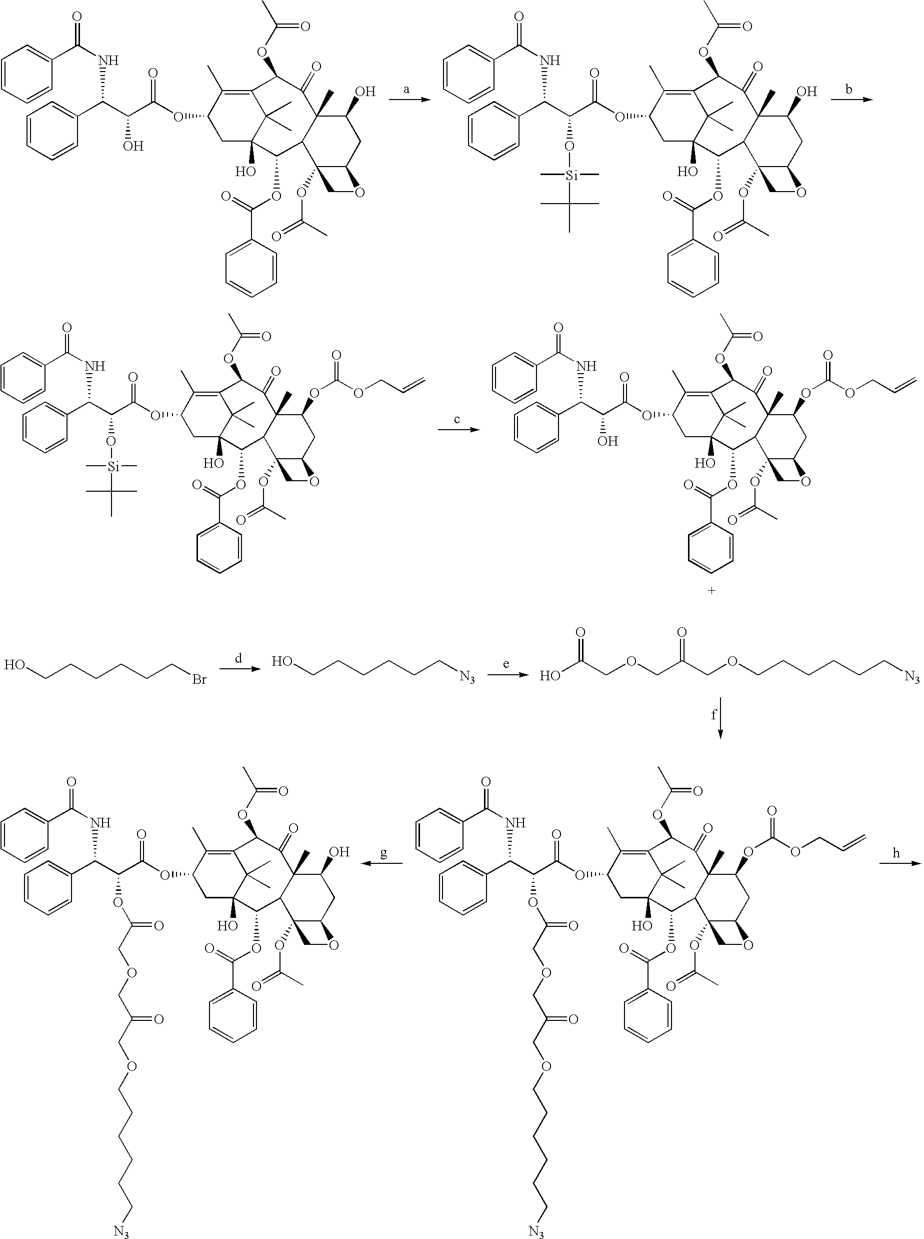

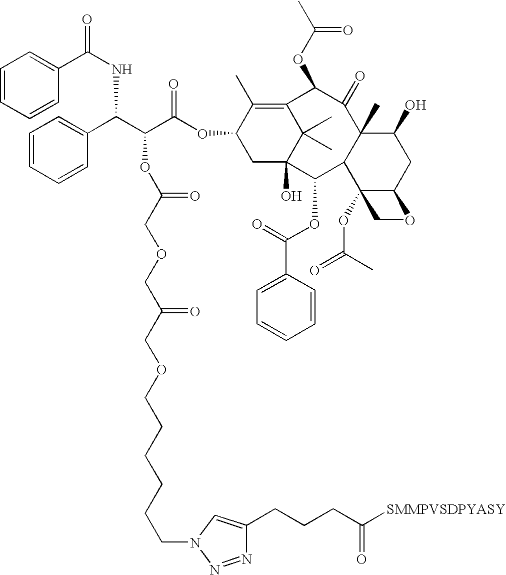

a) TBSCI/imidazole, THF, rt, 12 h; b) LiHMDS/allyl chloroformate, THF, −70° C., 1 h; c)TBAF, THF, rt, 3 h; d) NaN₃, DMF, rt, 24 h; e) diglycolic anhydride, THF, rt, 24 h; f) DIPC/azide-linker/DMAP, rt, 5 h; g)Pd(dba)₂/DPPE/2-thiobenzoic acid, it, 12 h; h) YSA motif, CuS0₄, sodium ascorbate, rt, 2 d.

### Example 3

### Synthesis of YSA Campothechin Conjugates (YSA-CPT)

Camptothecin (CPT) is a cytotoxic alkaloid which inhibits topoisomerase I. CPT was discovered in 1966 and showed remarkable anticancer activity in preliminary clinical trials. The drug however, presents low solubility and severe adverse reactions. Because of these disadvantages, a pro-drug version of the compound was proposed after years of research. As a result, two CPT analogues have been approved and are used in cancer chemotherapy today, including topotecan and irinotecan. Camptothecin-11 (irinotecan) is a semi-synthetic derivative of the natural alkaloid campothecin and its acts as chemotherapy agent by inhibiting topoisomerase 1. Currently, its main use in the clinic is in colon cancer, particularly in combination with other chemotherapy agents, but it is effective against a variety of other tumors. The adverse effects and poor solubility of this drug delayed its use in the United States until 1994, when Pfizer got Camptosar or CPT-11 approved by the FDA. Hence, while this appears as very useful drug, its solubility and adverse effects have been limiting factors. The design of YSA-delivering-CPT is therefore appealing. Using the linker and click chemistry described herein, the YSA-CPT derivatives are shown below.

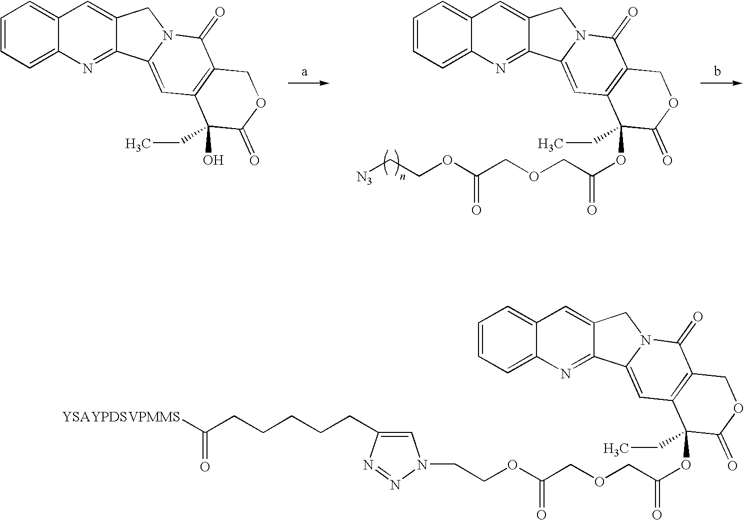

a) EDCI, azide acid, DMAP, DCM, rt, 85%, 24 h; b) CuS0₄, sodium ascorbate, MeOH, YSA motif, rt, 2 d, 75%.

### Example 4

### Efficacy of Compounds

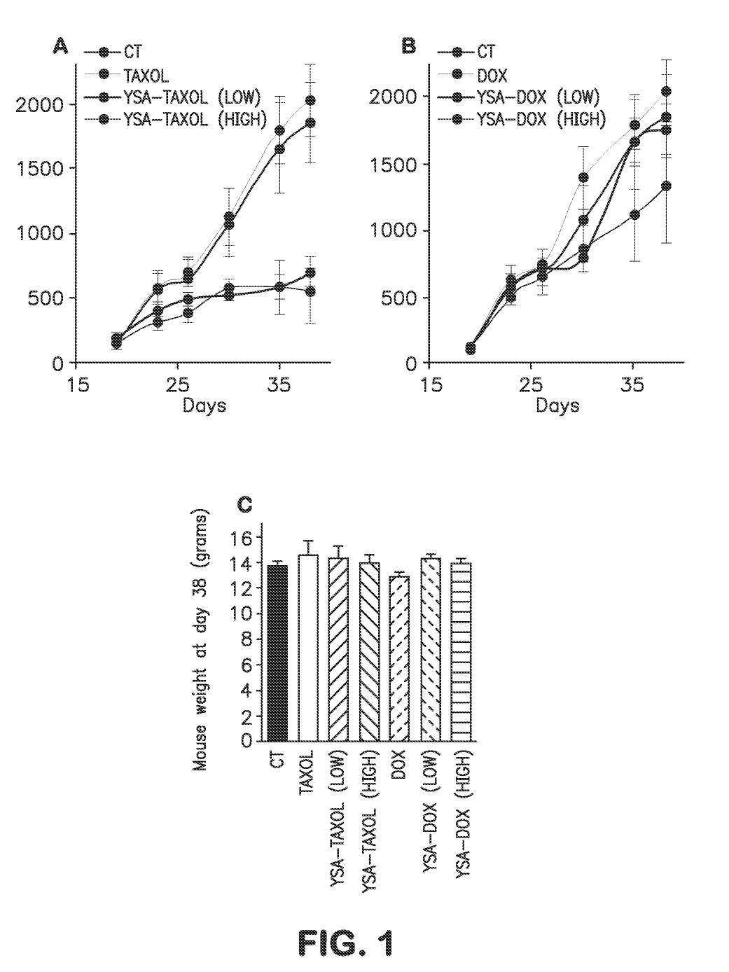

To evaluate the effects of the YSA-doxorubicin and YSA-taxol conjugates on tumor growth, PC3 tumor-bearing mice were treated twice a week with an i.v. injection of each drug for a total of ˜3 weeks (FIG. 1). Remarkable tumor growth inhibition was observed with both doses of YSA-taxol, while unconjugated taxol at the same molar dose was not significantly different from vehicle control. YSA-doxorubicin and unconjugated doxorubicin were also not significantly different from vehicle control (FIGS. 1A, B). Remarkably, 3 of the tumors treated with the high concentration of YSA-taxol and 2 of the tumors treated with the high concentration of YSA-doxorubicin could no longer be detected at days 35 and 38, after having initially grown to >500 nm (not shown). Moreover, mouse body weights were unaffected in all treatment groups, suggesting that YSA coupled to taxol or doxorubicin is not particularly toxic over a period of several weeks (FIG. 1C).

As shown in FIG. 1, YSA-taxol is more effective than taxol and doxorubicin in inhibiting tumor growth in vivo without showing toxicity. Groups of 5-7 SCID-beige mice bearing pre-established subcutaneous PC3 tumors were treated with twice weekly with i.v. doses of vehicle (CT), taxol (5 mg/Kg), doxorubicin (2 mg/Kg), YSA-taxol (at doses that are 1 and 5 fold molar equivalents of the taxol dose), YSA-doxorubicin (at doses that are 1 and 5 fold molar equivalents of the doxorubicin dose) starting at day 19. Tumor sizes were measured and averages +/−SE are shown. The data in (A) and (B) are from the same experiment and share the same control. Mouse weights from the day of the last measurement (day 38) are shown in (C), p<0.05 for the comparison of YSA-taxol (low) and YSA-taxol (high) with control, by repeated measures two-way ANOVA using all the measurements as well as by one-way ANOVA and Dunnett's posthoc test using the measurements at day 38. All other treatments were not significantly different from the control. Mouse weights in (C) were not significantly different from the control for all the treatments.

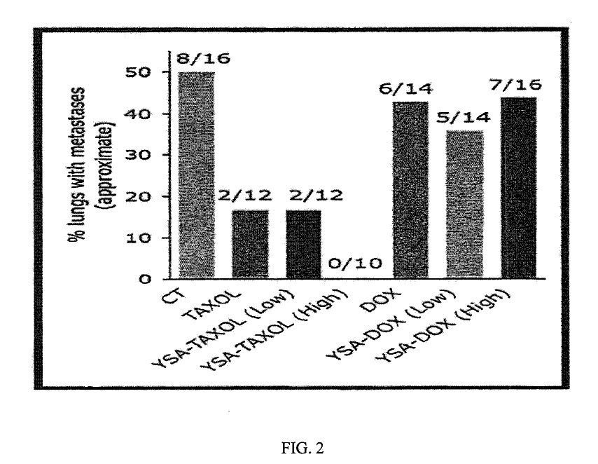

A number of factors, including the nature of the peptide, the nature of the linker, and the conjugated drug itself are all parameters that influence the potency and pharmacokinetics and distribution of the compound in vivo. It is contemplated that the plasma stability may be improved even further. It is also contemplated that measuring PK behavior of the optimized drugs in vivo may be accomplished to fine tune dose and regimen of the drugs when measuring efficacy in vivo. Surprisingly, the mice treated with YSA-TAX had virtually no lung metastases. FIG. 2 demonstrates the measurements of lung metastases in PC3L xenograft mice treated with various agents including CT, Taxol, YSA-taxol (low), YSA-taxol (high), DOX, YSA-DOX (low), and YSA-DOX (high).

While the present disclosure has been particularly shown and described with reference to several embodiments thereof, it will be understood by those of ordinary skill in the art that various changes in form and details may be made thereto without departing from the principles and spirit of the present disclosure, the proper scope of which is defined in the following claims and their equivalents.

What is claimed is:

1. A compound of Formula I:
EPH_T-L-D (I),
or a pharmaceutically acceptable salt, solvate or hydrate thereof, wherein:
EPH-T is an Eph receptor binding compound;
L is a linking group; and
D is a chemotherapeutic agent.

2. The compound of claim 1, wherein the EPH-T is a YSA peptide having Formula II: 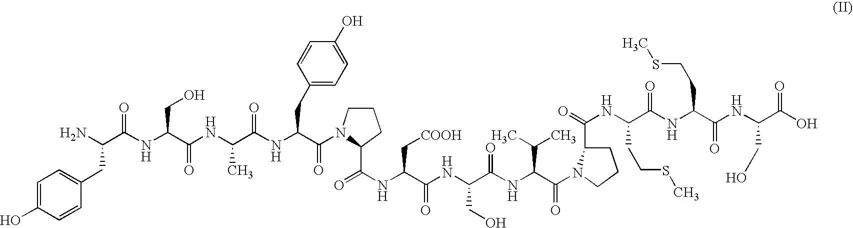
or amino acid sequence YSAYPDSVPMMS, wherein Y is tyrosine; S is serine; A is alanine; P is proline; D is aspartic acid; V is valine; and M is methionine.

3. The compound of claim 2, wherein the YSA peptide having Formula II or amino acid sequence YSAYPDSVPMMS, is substituted with any of the amino acid substitutions as follows:
each Y is optionally S, T, C, N or Q;
each S is optionally T, C, Y, N or Q;
each A is optionally G, V, L, nL, I, M, F, W or P;
each P is optionally G, A, V, L, nL, I, M, F or W;
each D is optionally E;
each V is optionally G, A, L, nL, I, M, F, W or P; and
each M is optionally G, A, V, L, nL, I, F, W or P,
wherein: G is glycine; A is alanine; V is valine; L is leucine; nL is nor-Leucine; I is isoleucine; M is methionine; F is phenylalanine; W is tryptophan; P is proline; S is serine; T is threonine; C is cysteine; Y is tyrosine; N is asparagine; Q is glutamine; D is aspartic acid; E is glutamic acid; K is lysine; R is arginine; and H is histidine.

4. The compound of claim 2, wherein the YSA peptide having Formula II or amino acid sequence YSAYPDSVPMMS, has amino acid sequence: YSAYPDSVPnLnLS, wherein nL is nor-Leucine.

5. The compound of claim 1, wherein the linking group has Formula III or IV: 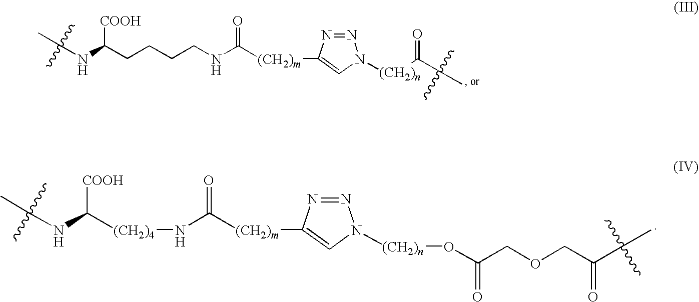
wherein m and n are each independently integers from 0, 1, 2, 3, 4, 5, 6, 7, 8, 9, 10, 11, or 12.

6. The compound of claim 5, wherein m is 2; and n is 2.

7. The compound of claim 1, wherein the chemotherapeutic agent is taxol, doxorubicin, taxotere, campotechin, or etoposide.

8. The compound of claim 1, wherein the compound of Formula I has Formula V: 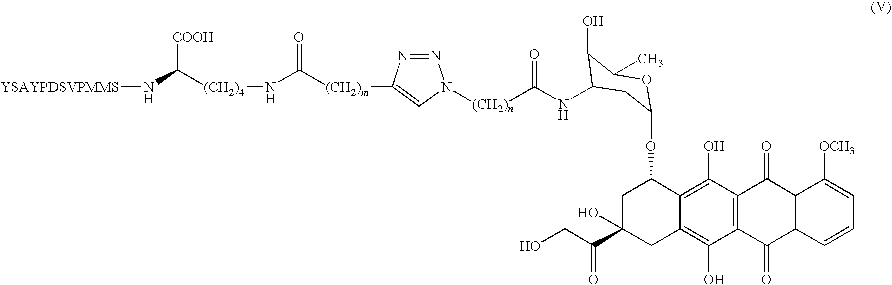
wherein m and n are each independently an integer from 0, 1, 2, 3, 4, 5, 6, 7, 8, 9, 10, 11, or 12.

9. The compound of claim 1, wherein the compound of Formula has Formula VI:

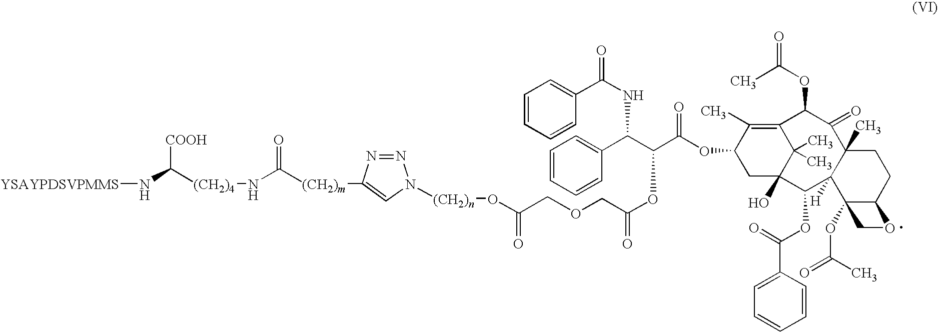

10. The compound of claim 1, wherein the compound of Formula I has Formula VII:

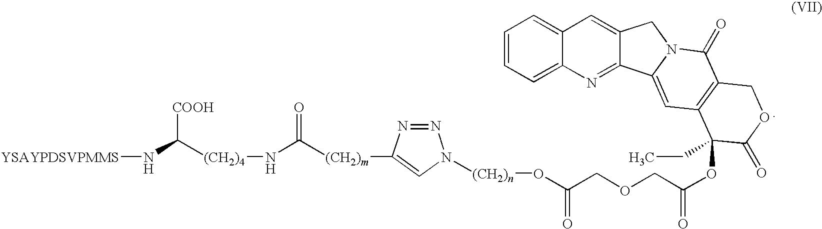

11. A compound of Formula VIII: 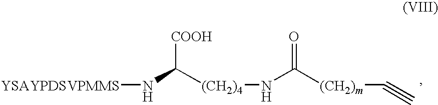
or a pharmaceutically acceptable salt, solvate or hydrate thereof, wherein m is independently an integer from 0, 1, 2, 3, 4, 5, 6, 7, 8, 9, 10, 11, or 12.

12. A compound of Formula IX: 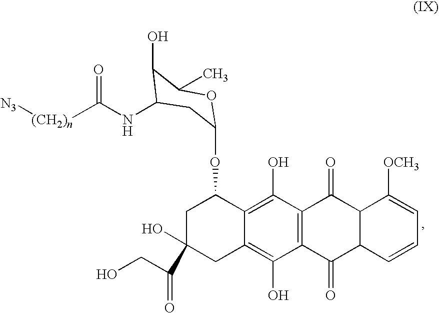
or a pharmaceutically acceptable salt, solvate or hydrate thereof, wherein n is independently an integer from 0, 1, 2, 3, 4, 5, 6, 7, 8, 9, 10, 11, or 12.

13. A compound of formula X: 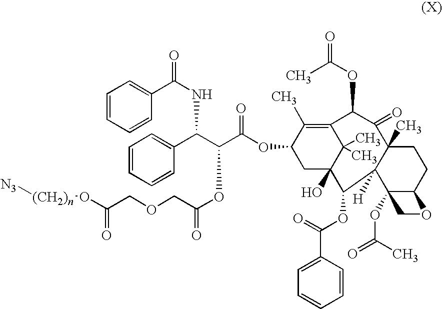
or a pharmaceutically acceptable salt, solvate or hydrate thereof, wherein n is independently an integer from 0, 1, 2, 3, 4, 5, 6, 7, 8, 9, 10, 11, or 12.

14. A compound of formula XI: 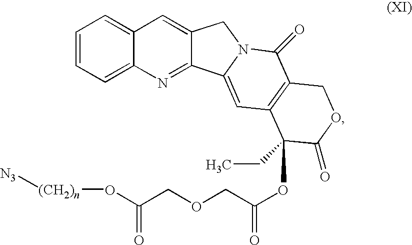
or a pharmaceutically acceptable salt, solvate or hydrate thereof, wherein n is independently an integer from 0, 1, 2, 3, 4, 5, 6, 7, 8, 9, 10, 11, or 12.

15. A pharmaceutical composition comprising the compound of Formula I of claim 1, and a pharmaceutically acceptable solvent.

16. A method of treating cancer, the method comprising the steps of administering a pharmacologically effective amount of the pharmaceutical composition of claim 15 to a patient in need thereof.

17. A method of targeting delivery of chemotherapeutic agents to the Eph receptor, the method comprising the steps of contacting the Eph receptor with the compound of Formula I of claim 1.

18. A method of preparing the compound of Formula I of claim 1:
EPH_T-L-D (I),
the method comprising the steps of
a) coupling the EPH_T (Eph receptor binding compound) to an alkynoic acid;
b) coupling the D (chemotherapeutic agent) to an azide; and
c) reacting the EPH-T (Eph receptor binding compound) coupled to an alkynoic acid with the D (chemotherapeutic agent) coupled to an azide in a 1,3-dipolar cycloaddition reaction to form a 1,4-disubstituted-1,2,3-triazole compound of Formula I.

19. The method of claim 18, wherein the EPH-T is a YSA peptide having Formula II: 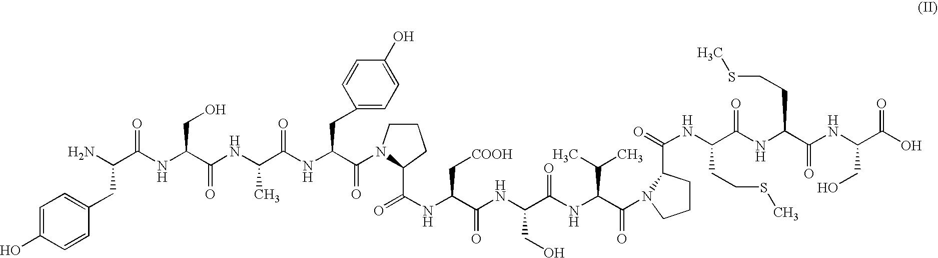
or amino acid sequence YSAYPDSVPMMS, wherein Y is tyrosine; S is serine; A is alanine; P is proline; D is aspartic acid; V is valine; and M is methionine.

20. The method of claim 19, wherein the YSA peptide having Formula II or amino acid sequence YSAYPDSVPMMS, is substituted with any of the amino acid substitutions as follows:
each Y is optionally S, T, C, N or Q;
each S is optionally T, C, Y, N or Q;
each A is optionally G, V, L, nL, I, M, F, W or P;
each P is optionally G, A, V, L, nL, I, M, F or W;
each D is optionally E;
each V is optionally G, A, L, nL, I, M, F, W or P; and
each M is optionally G, A, V, L, nL, I, F, W or P,
wherein: G is glycine; A is alanine; V is valine; L is leucine; nL is nor-Leucine; I is isoleucine; M is methionine; F is phenylalanine; W is tryptophan; P is proline; S is serine; T is threonine; C is cysteine; Y is tyrosine; N is asparagine; Q is glutamine; D is aspartic acid; E is glutamic acid; K is lysine; R is arginine; and H is histidine.

21. The method of claim 19, wherein the YSA peptide having Formula II or amino acid sequence YSAYPDSVPMMS, has amino acid sequence: YSAYPDSVPnLnLS, wherein nL is nor-Leucine.

22. The method of claim 18, wherein the 1,4-disubstituted-1,2,3-triazole compound has Formula III or IV: 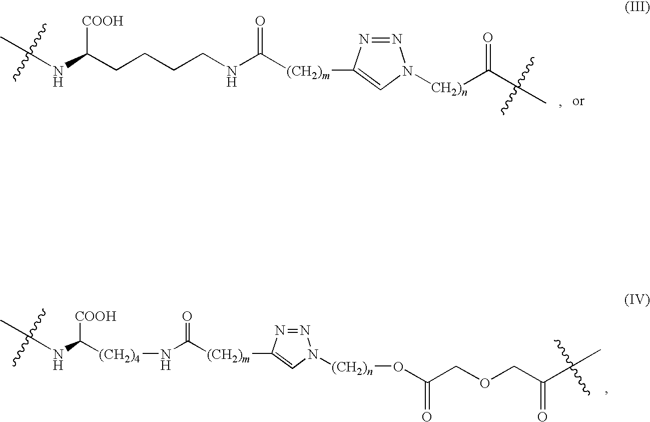
wherein m and n are each independently integers from 0, 1, 2, 3, 4, 5, 6, 7, 8, 9, 10, 11, or 12.

23. The method of claim 22, wherein m is 2; and n is 2.

24. The method of claim 18, wherein the chemotherapeutic agent is taxol, doxorubicin, taxotere, campotechin, or etoposide.

25. The method of claim 18, wherein the compound of Formula I has Formula V: 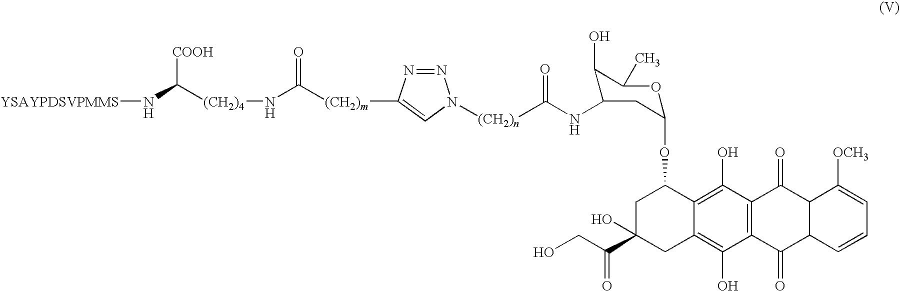
wherein m and n are each independently an integer from 0, 1, 2, 3, 4, 5, 6, 7, 8, 9, 10, 11, or 12.

26. The method of claim 18, wherein the compound of Formula I has Formula VI:

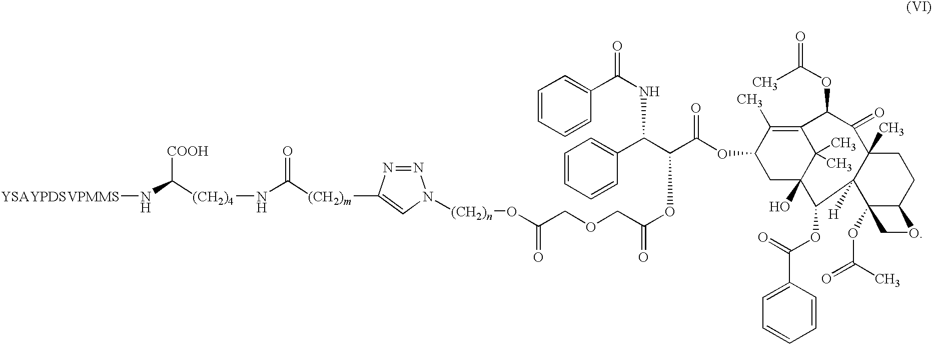

27. The method of claim 18, wherein the compound of Formula I has Formula VII:

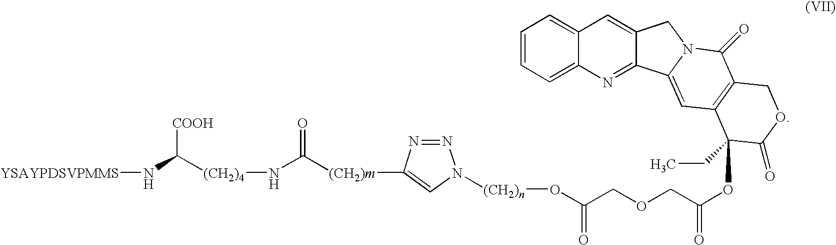

28. The method of claim 18, wherein the coupling of the EPH_T (Eph receptor binding compound) to an alkynoic acid provides a compound of Formula VIII: 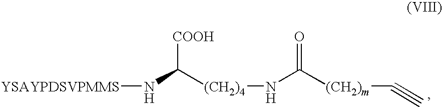
or a pharmaceutically acceptable salt, solvate or hydrate thereof, wherein m is independently an integer from 0, 1, 2, 3, 4, 5, 6, 7, 8, 9, 10, 11, or 12.

29. The method of claim 18, wherein the coupling of the D (chemotherapeutic agent) to an azide provides a compound of Formula IX: 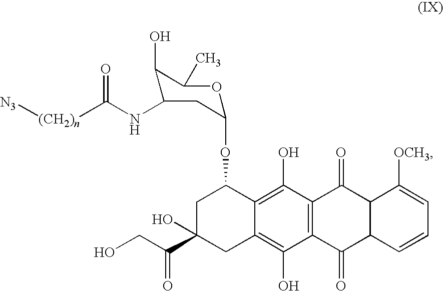
or a pharmaceutically acceptable salt, solvate or hydrate thereof, wherein n is independently an integer from 0, 1, 2, 3, 4, 5, 6, 7, 8, 9, 10, 11, or 12.

30. The method of claim 18, wherein the coupling of the D (chemotherapeutic agent) to an azide provides a compound of formula X: 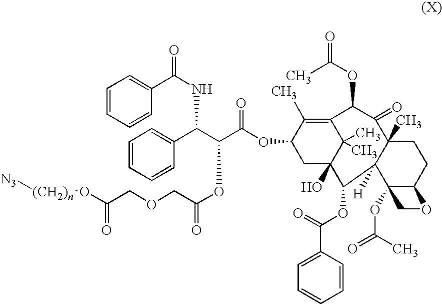
or a pharmaceutically acceptable salt, solvate or hydrate thereof, wherein n is independently an integer from 0, 1, 2, 3, 4, 5, 6, 7, 8, 9, 10, 11, or 12.

31. The method of claim 18, wherein the coupling of the D (chemotherapeutic agent) to an azide provides a compound of formula XI: 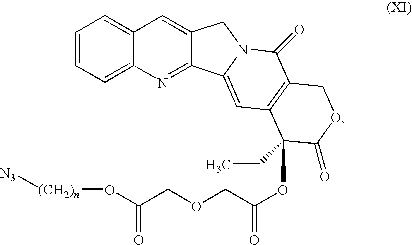
or a pharmaceutically acceptable salt, solvate or hydrate thereof, wherein n is independently an integer from 0, 1, 2, 3, 4, 5, 6, 7, 8, 9, 10, 11, or 12.
